(19) United States
(12) Patent Application Publication
(10) Pub. No.: US 2017/0086860 A1
(43) Pub. Date: Mar. 30, 2017

(54) SUPPORT CATHETER AND GUIDEWIRE KIT FOR CROSSING A VASCULAR OCCLUSION

(71) Applicant: The Spectranetics Corporation, Colorado Springs, CO (US)

(72) Inventor: Weston H. LEE, Colorado Springs, CO (US)

(21) Appl. No.: 15/274,181

(22) Filed: Sep. 23, 2016

Related U.S. Application Data
(60) Provisional application No. 62/233,121, filed on Sep. 25, 2015.

(51) Int. Cl. A61B 17/22; A61B 17/12; A61B 18/24

(52) U.S. Cl. CPC A61B 17/22; A61B 18/245; A61B 17/12145; A61B 17/12131; A61B 2017/22094; A61B 2017/22042; A61B 2017/22001; A61B 2018/00577

(57) ABSTRACT

The present disclosure relates generally to the use of medical devices for the treatment of vascular conditions. In particular, the present disclosure provides devices and methods of using a kit, which includes a guidewire and a support catheter, both of which are capable of penetrating a calcified and/or fibrous total occlusion. The guidewire and the support catheter each comprise an expandable member adjacent its distal end. The guidewire and support catheter are used in cooperation with one another to cross an occlusion in a step-wise fashion by alternately deploying and retracting the expandable member of the support catheter while the guidewire is inserted into the occlusion and deploying the expandable member of the guidewire while the support catheter is inserted into the occlusion.

### CROSS REFERENCE TO RELATED APPLICATIONS

The present application claims the benefit of and priority to, under 35 U.S.C. §119(e), U.S. Provisional Application Ser. No. 62/233,121, filed Sep. 25, 2015, entitled “SUPPORT CATHETER AND GUIDEWIRE KIT FOR CROSSING A VASCULAR OCCLUSION,” the disclosure of which is hereby incorporated by reference in its entirety for all that it teaches and for all purposes.

### FIELD

The present disclosure relates generally to the use of medical devices for the treatment of vascular conditions. In particular, the present disclosure provides devices and methods for crossing a vascular occlusion using a support catheter and guidewire kit.

### BACKGROUND

Arterial disease is a common disease that affects millions of Americans. Coronary artery disease (CAD) most often results from a condition known as atherosclerosis, which generally manifests as the accumulation of a waxy substance on the inside of a subject's coronary arteries. This substance, called plaque, is made of cholesterol, fatty compounds, calcium, and a blood-clotting material called fibrin. Similarly, peripheral artery disease (PAD) often results from the accumulation of plaque on the inside of a subject's peripheral arteries, such as the arteries in a patient's arms, hands, legs and/or feet. As the plaque builds up, the artery narrows, or becomes stenotic, making it more difficult for blood to flow through the arteries. As the size of the stenosis increases and the blockage worsens, blood flow slows and upon the formation of a total occlusion, blood flow through the corresponding artery completely stops. Over time, an occlusion, particularly a total occlusion, may calcify and/or becomes fibrous, thereby increasing the balloon's ability to dilate the occlusion.

Atherectomy devices, such as mechanical cutting or removal devices, such as orbital, rotational, and directional mechanical, as well as ablation devices, such as laser ablation or radio-frequency ablation devices, are typically used in conjunction with a guidewire to remove the undesirable occlusion. For example, various laser atherectomy catheters are sold by The Spectranetics Corporation under the tradename ELCA™, which is used for coronary vascular intervention, and Turbo Elite™ which is used for peripheral vascular intervention, both of which perform catheterization such as recanalizing occluded arteries, changing lesion morphology, and facilitating stent placement. The atherectomy devices generally have a lumen through them, thereby allowing a guidewire to pass though the atherectomy devices. The guidewire is often first introduced into a patient's vasculature, and the guidewire acts as a rail, over which the atherectomy device can be inserted into vasculature. It is desirable for the guidewire to cross the occlusion prior to the atherectomy device so the atherectomy device can follow the guidewire's path through the occlusion.

In the event an occlusion has become calcified and/or fibrous, it may be difficult for the guidewire to pierce and cross the occlusion. Specifically, when a clinician attempts to push the guidewire into the calcified occlusion, the guidewire may bend and/or buckle due to an insufficient amount of rigidity. A support catheter, such as the support catheter sold by The Spectranetics Corporation under the tradename QUICK-CROSS™ which is used for vascular intervention, may be used in conjunction with the guidewire to reduce the guidewire's likelihood of bending or buckling. Referring to FIG. 1, there is depicted the distal portion of a guidewire 112 surrounded by a support catheter 116, both of which are within the patient's vasculature 108 adjacent an occlusion 104. Depending upon the hardness of the calcified occlusion, the combination of both the support catheter 116 and guidewire 112 may still not provide the guidewire with sufficient stiffness to pierce and/or cross the calcified occlusion.

### SUMMARY

What is needed is a system or kit, which includes a guidewire and a support catheter, which are capable of penetrating a calcified and/or fibrous total occlusion. These and other needs are addressed by the various aspects, embodiments, and configurations of the present disclosure.

The present disclosure provides a kit comprising a catheter having a proximal end and a distal end, wherein the catheter comprises an expandable member adjacent its distal end, and a guidewire having a proximal end and a distal end, wherein the guidewire is configured to be disposed radially within the catheter, and wherein the guidewire comprises an expandable member adjacent its distal end.

A kit according to paragraph [0007], wherein the expandable member of the catheter comprises at least one of an expandable braided element, an expandable coiled wire, pivotable barbs, a radially expandable hypotube, and expandable ribbon.

A kit according to any of paragraphs [0007]-[0008], wherein the expandable member of the guidewire comprises at least one of an expandable braided element, an expandable coiled wire, pivotable barbs, a radially expandable hypotube, and expandable ribbon.

The present disclosure also provides a method of crossing an occlusion within the vasculature of a subject, the method comprising introducing a kit within the vasculature of the subject, the kit comprising a catheter having a proximal end and a distal end, wherein the catheter comprises an expandable member adjacent its distal end, and a guidewire having a proximal end and a distal end, wherein the guidewire is configured to be disposed radially within the catheter, and wherein the guidewire comprises an expandable member adjacent its distal end, positioning the guidewire and the catheter adjacent the occlusion within the vasculature, advancing the guidewire into the occlusion, expanding the expandable member of the guidewire into the occlusion, advancing the catheter into the occlusion over the guidewire, expanding the expandable member of the catheter into the occlusion, retracting the expandable member of the guidewire from the occlusion, and advancing the guidewire through the occlusion.

A method according to paragraph [0010], further comprising the step of removing the catheter from the vasculature.

A method according to paragraphs [0010]-[0011], wherein the expandable member of the catheter comprises at least one of an expandable braided element, an expandable coiled wire, pivotable barbs, a radially expandable hypotube, and expandable ribbon.

A method according to paragraphs [0010]-[0011], wherein the expandable member of the guidewire comprises at least one of an expandable braided element, an expandable coiled wire, pivotable barbs, a radially expandable hypotube, and expandable ribbon.

The present disclosure also provides a method of removing a portion of an occlusion within a vasculature of a subject, the method comprising introducing a kit within the vasculature of the subject, the kit comprising a catheter having a proximal end and a distal end, wherein the catheter comprises an expandable member adjacent its distal end, and a guidewire having a proximal end and a distal end, wherein the guidewire is configured to be disposed radially within the catheter, wherein the guidewire comprises an expandable member adjacent its distal end, positioning the guidewire and the catheter adjacent the occlusion within the vasculature, advancing the guidewire into the occlusion, expanding the guidewire's expandable member into the occlusion, advancing the catheter into the occlusion over the guidewire, expanding the expandable member of the catheter into the occlusion, retracting the expandable member of the guidewire from the occlusion, advancing the guidewire through the occlusion, removing the catheter from the vasculature, introducing an atherectomy device into the vasculature over the guidewire and removing the portion of the occlusion from the vasculature with the atherectomy device.

A method according to paragraph [0014], wherein the atherectomy device is a laser ablation catheter.

A method according to paragraphs [0014]-[0015], wherein the expandable member of the catheter comprises at least one of an expandable braided element, an expandable coiled wire, pivotable barbs, a radially expandable hypotube, and expandable ribbon.

A method according to paragraphs [0014]-[0016], wherein the expandable member of the guidewire comprises at least one of an expandable braided element, an expandable coiled wire, pivotable barbs, a radially expandable hypotube, and expandable ribbon.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “catheter” as used herein generally refers to a tube that can be inserted into a body cavity, duct, lumen, or vessel, such as the vasculature system. In most uses, a catheter is a relatively thin, flexible tube (“soft” catheter), though in some uses, it may be a larger, solid-less flexible—but possibly still flexible—catheter (“hard” catheter).

A “coupler” or “fiber optic coupler” refers to the optical fiber device with one or more input fibers and one or several output fibers. Fiber couplers are commonly special optical fiber devices with one or more input fibers for distributing optical signals into two or more output fibers. Optical energy is passively split into multiple output signals (fibers), each containing light with properties identical to the original except for reduced amplitude. Fiber couplers have input and output configurations defined as M×N. M is the number of input ports (one or more). N is the number of output ports and is always equal to or greater than M. Fibers can be thermally tapered and fused so that their cores come into intimate contact. This can also be done with polarization-maintaining fibers, leading to polarization-maintaining couplers (PM couplers) or splitters. Some couplers use side-polished fibers, providing access to the fiber core. Couplers can also be made from bulk optics, for example in the form of micro lenses and beam splitters, which can be coupled to fibers (“fiber pig-tailed”).

The terms “emitter” as used herein refers to an end portion of a fiber or an optical component that emits light from a distal end of device, such as a catheter, towards a desired target or region, which typically comprises tissue. As described herein, an emitter or emitters can be used to emit light of any wavelength, insofar as the light emitted is coupled with a suitable absorptive liquid such that pressure waves and/or cavitation bubbles are generated and shock waves are produced. An emitter or emitters can emit light, including but not limited to, laser light, white light, visible light, infrared light, and ultraviolet light.

A “laser emitter” as used herein refers to an end portion of a fiber or an optical component that emits laser light from a distal end of the catheter towards a desired target, which is typically tissue.

An optical fiber (or laser active fibre) as used herein refers to a flexible, transparent fiber made of an optically transmissive material, such as glass (silica) or plastic, which functions as a waveguide, or “light pipe”, to transmit light between the two ends of the fiber.

The terms “vasculature” and “vascular” as used herein refer to any part of the circulatory system of a subject, including peripheral and non-peripheral arteries (e.g., coronary) and veins. Vascular material found within the vasculature can be comprised of both biological material (e.g., nucleic acids, amino acids, carbohydrates, polysaccharides, lipids and the like) and non-biological material (e.g., fat deposits, fibrous tissue, calcium deposits, remnants of dead cells, cellular debris and the like).

The term “means” as used herein shall be given its broadest possible interpretation in accordance with 35 U.S.C. §112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 illustrates an elevation view of a prior art kit within the vasculature of a patient, wherein the kit includes a guidewire radially disposed within a support catheter, which is over the guidewire, wherein the support catheter and guidewire are located proximally of an occlusion;

FIG. 2 is an elevation view of a kit within the vasculature of a patient, wherein the kit includes a guidewire radially disposed within a support catheter, which is over the guidewire, according to one embodiment of the present disclosure, wherein the support catheter and guidewire are located proximally of an occlusion;

FIG. 3 is an elevation view of a kit within the vasculature of a patient, wherein the kit includes a guidewire radially disposed within a support catheter, which is over the guidewire, according to one embodiment of the present disclosure, wherein the support catheter is located proximally of an occlusion, the guidewire has penetrated the occlusion, and the guidewire's expandable member is in a unexpanded state;

FIG. 4 is an elevation view of a kit within the vasculature of a patient, wherein the kit includes a guidewire radially disposed within a support catheter, which is over the guidewire, according to one embodiment of the present disclosure, wherein the support catheter is located proximally of an occlusion, the guidewire has penetrated the occlusion, and the guidewire's expandable member is in an expanded state;

FIG. 5 is an elevation view of a kit within the vasculature of a patient, wherein the kit includes a guidewire radially disposed within a support catheter, which is over the guidewire, according to one embodiment of the present disclosure, wherein the support catheter and the guidewire have penetrated the occlusion, the guidewire's expandable member is in an expanded state, and the support catheter's expandable member is in a unexpanded state;

FIG. 6 is an elevation view of a kit within the vasculature of a patient, wherein the kit includes a guidewire radially disposed within a support catheter, which is over the guidewire, according to one embodiment of the present disclosure, wherein the support catheter and the guidewire have penetrated the occlusion, the guidewire's expandable member is in an unexpanded state, and the support catheter's expandable member is in an expanded state;

FIG. 7 is an elevation view of guidewire, according to one embodiment of the present disclosure, wherein the guidewire has an expandable member is in an unexpanded state;

FIG. 7A is a cross-sectional view of the guidewire of FIG. 7, wherein the guidewire has an expandable member is in an unexpanded state;

FIG. 7B is a cross-sectional view of the guidewire of FIG. 7, wherein the guidewire has an expandable member is in an expanded state;

FIG. 8A is a cross-sectional view of guidewire, according to one embodiment of the present disclosure, wherein the guidewire has an expandable member in an un-extended state;

FIG. 8B is a cross-sectional view of guidewire of FIG. 8A, according to one embodiment of the present disclosure, wherein the guidewire has an expandable member in an extended state;

FIG. 9 is a cross-sectional view of guidewire, according to one embodiment of the present disclosure, wherein the guidewire has an expandable member in an unexpanded state;

FIG. 10A is a cross-sectional view of a guidewire, wherein the guidewire has an expandable member is in an unexpanded state;

FIG. 10B is a cross-sectional view of the guidewire of FIG. 10A, wherein the guidewire has an expandable member is in an expanded state; and

FIG. 11 is a representative flow diagram of a method of treating a subject using kit, which includes a guidewire and a support catheter, and a laser atherectomy catheter, according to one embodiment of the present disclosure.

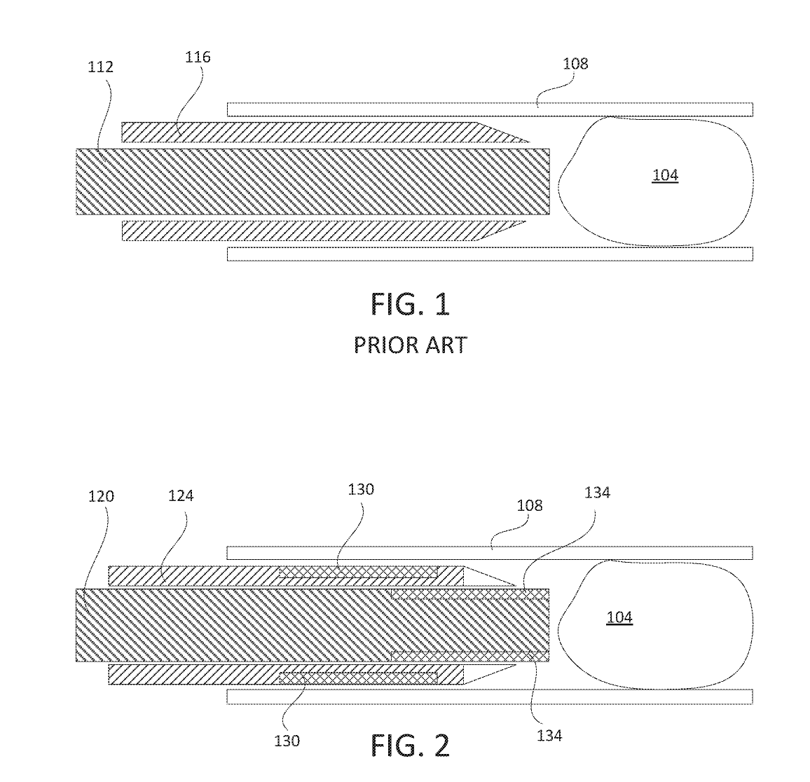

### DETAILED DESCRIPTION

The present disclosure relates generally to the use of medical devices for the treatment of vascular conditions. In particular, the present disclosure provides materials and methods for using laser-induced shock waves to disrupt vascular blockages and to deliver therapeutic agents to the blockage area.

Referring to FIG. 2, there is depicted an exemplary kit including a guidewire 120 radially disposed within a support catheter 124, which is over the guidewire 120, wherein the support catheter 124 and guidewire 120 are located proximally of an occlusion 104 within a patient's vasculature 108. The guidewire 120 includes an expandable member 134 at, proximate or adjacent its distal end. The support catheter 124 also includes an expandable member 130, which may also be referred to as an engagement member, at, proximate or adjacent its distal end. The expandable members 130, 134 may be the same or different.

The guidewire 120 and support catheter 124 are used in cooperation with one another to cross an occlusion, particularly a calcified and/or fibrous occlusion, in a step-wise fashion. Specifically, engagement of expandable member 130 of the support catheter 124 and the occlusion 104 allows the support catheter 124 to remain relatively stationary within the occlusion 104 as the guidewire 120 is inserted into the occlusion, thereby increasing the rigidity and column strength of the guidewire 120 and reducing the possibility of the support catheter 124 from backing out of the vasculature and/or away from the occlusion 104 during insertion of the guidewire 120. Similarly, engagement of expandable member 134 of the guidewire 120 and the occlusion 104 allows the guidewire 120 to remain relatively stationary within the occlusion 104 as the support catheter 124 is inserted into the occlusion, thereby reducing the possibility of the guidewire 120 from backing out of the vasculature and/or away from the occlusion 104 during insertion of the support catheter 124.

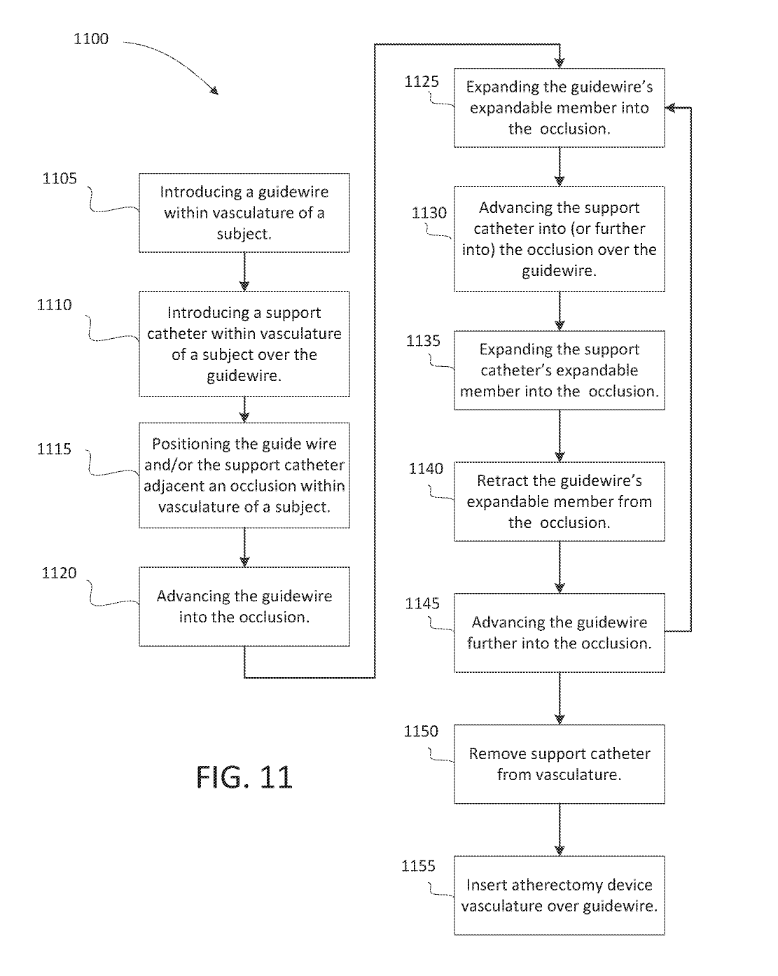

Referring to FIG. 11 there is depicted a representative flow diagram of a method of treating a subject using the kit, which includes the guidewire 120, the support catheter 124, and an atherectomy catheter (not shown) to remove at least a portion of the occlusion 104 after the guidewire 120 and/or the support catheter 124 have crossed the occlusion 104. As discussed herein above, atherectomy devices may include mechanical cutting or removal devices, such as orbital, rotational, and directional mechanical devices, as well as ablation devices, such as laser ablation or radio-frequency ablation devices may be used in conjunction with the guidewire 120 to remove the undesirable occlusion.

Although it is not illustrated in this disclosure, an ablation system typically includes a laser apparatus coupled to a laser controller. The controller includes one or more computing devices programmed to control a laser. The controller may be internal or external to the laser apparatus, such as a laser generator. The laser apparatus may include an excimer laser or another suitable laser. In some embodiments, the laser produces light in the ultraviolet frequency range, and in a certain embodiment, the laser may produce optical energy in pulses.

The laser is connected with the proximal end of a laser energy delivery system, which generally comprises a laser catheter, via a coupler. The laser catheter includes one or more transport members, such as optical fibers, which receive laser energy from the laser and transport the received laser energy from a proximal end of laser catheter towards a distal end of the laser catheter. The distal end of the laser catheter may be inserted into a vessel or tissue of a human body toward a target area, such as an occlusion, in human body.

Exemplary laser catheter devices or assemblies may include laser catheters and/or laser sheaths. Examples of laser catheters or laser sheath are sold by The Spectranetics Corporation under the tradenames ELCA™ and Turbo Elite™ (each of which is used for coronary intervention or peripheral intervention, respectively, such as recanalizing occluded arteries, changing lesion morphology, and facilitating stent placement). The working (distal) end of a laser catheter typically has a plurality of laser emitters that emit energy and ablate the targeted tissue. The opposite (proximal) end of a laser catheter typically has a fiber optic coupler. The fiber optic coupler connects to a laser system or generator such as the CVX-300 Excimer Laser System, which is also sold by The Spectranetics Corporation.

The laser controller includes a non-transitory computer-readable medium (e.g., memory) that includes instructions that, when executed, cause one or more processors to control the laser and/or other components of ablation system. The controller includes one or more input devices to receive input from an operator. Exemplary input devices include keys, buttons, touch screens, dials, switches, mouse, and trackballs which providing user control of the laser. The controller further includes one or more output devices to provide feedback or information to an operator. Exemplary output devices include a display, lights, audio devices which provide user feedback or information.

Referring again to FIG. 11, the flowchart depicts a method 1100 that includes multiple steps, some or all of which may be used to remove a portion of the occlusion 104 within the vasculature 108. The method 1100 may comprise the step of introducing a kit within the vasculature 108 of a subject, wherein the kit comprises a support catheter 124 and a guidewire 120. FIG. 11 depicts the method 1100 including the individual step 1105 of introducing the guidewire 120 into the patient's vasculature and the individual step 1110 of introducing the support catheter 124 into the patient's vasculature. The steps 1105, 1110, however, may be performed serially or in parallel. That is, the steps 1105, 1110 may be performed individually, such that step 1105 is performed before step 1110 or step 1110 is performed before step 1105, or the steps 1105, 1110 may be performed simultaneously.

After the support catheter 124 and a guidewire 120 are inserted into the patient's vasculature, the support catheter 124 and/or the guidewire 120 are advanced adjacent the proximal side of the occlusion within the vasculature according to step 1115 of FIG. 11 and as depicted in FIG. 2. As discussed above, the guidewire 120 includes an expandable member 134 at, proximate or adjacent its distal end, and the support catheter 124 includes an expandable member 130 at, proximate or adjacent its distal end. FIG. 2 depicts the expandable members 130, 134 in a retracted or unexpanded state. However, when the support catheter 124 is adjacent the occlusion 104, its expandable member 130 may be expanded against or into the patient's vasculature 108.

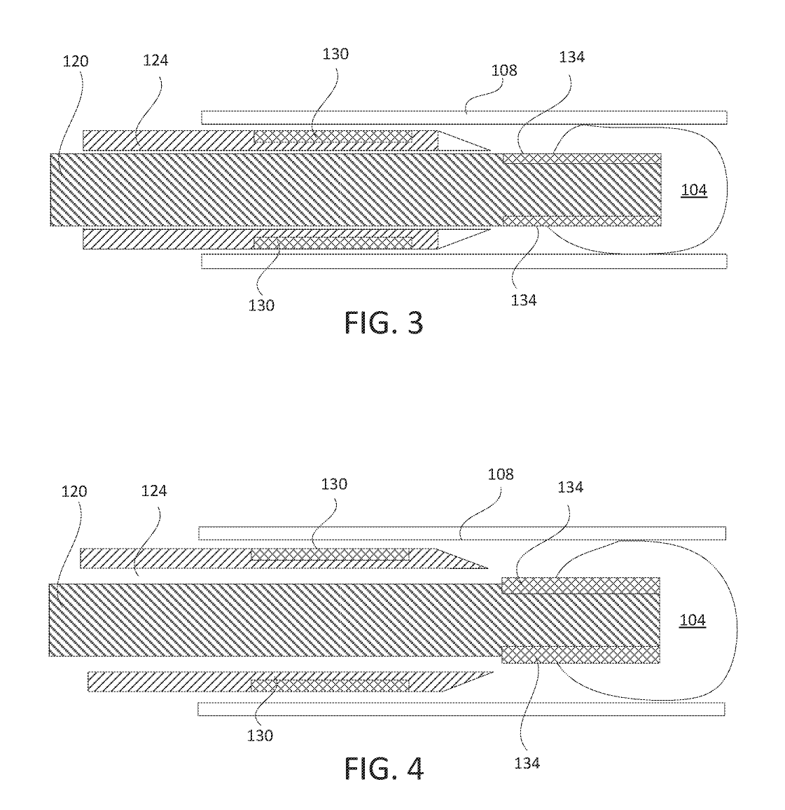

As the support catheter 124 remains adjacent the occlusion 104, the clinician pushes and advances the guidewire 120 into the occlusion 104 according to step 1120 of FIG. 11 and as depicted in FIG. 3. As the guidewire 120 advances into the occlusion 104, it may be desirable for its expandable member 134 to remain in its retracted or unexpanded state so as to increase the ease with which the guidewire 120 enters the occlusion 104. Again, as the guidewire 120 advances into the occlusion 104, the expandable member 130 of the support catheter 124 may be in its expanded or unexpanded state.

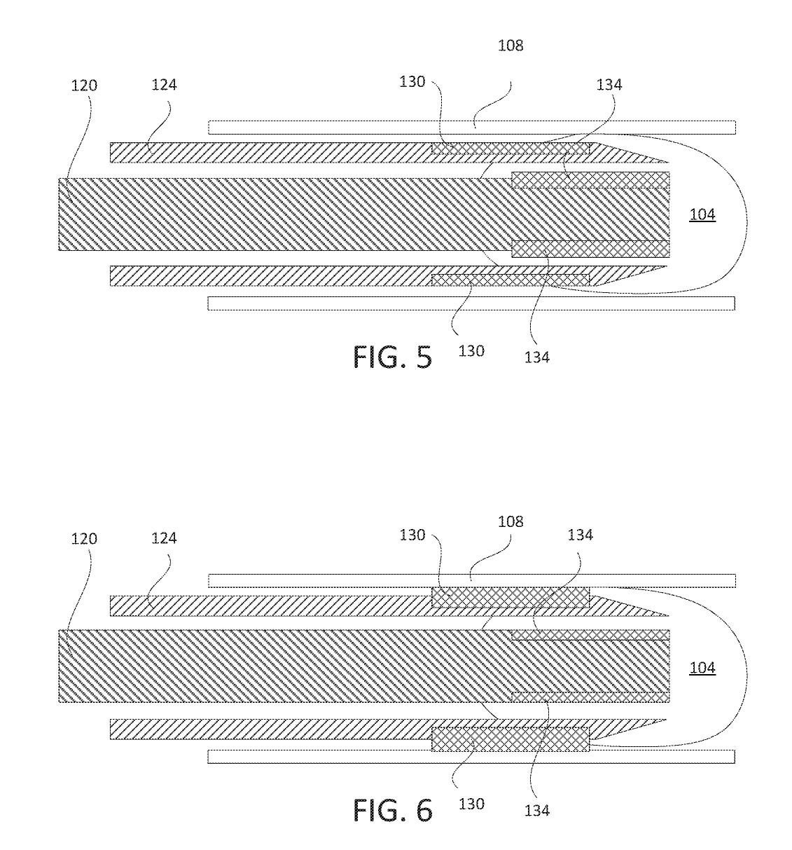

After the guidewire 120 has been inserted at least partially into the occlusion 104, it may be desirable to expand the guidewire's expandable member 134 into the occlusion 104, thereby locking the guidewire 120 within the occlusion, according to step 1125 of FIG. 11 and as depicted in FIG. 4. After the guidewire's expandable member 134 is expanded into the occlusion 104, the clinician may insert the support catheter 124 into the occlusion 104 over the guidewire 120 and over the guidewire's expandable member 134, according to step 1130 of FIG. 11 and as depicted in FIG. 5. Expanding the guidewire's expandable member 134 into the occlusion 104 assists in holding the guidewire 120 in place during insertion of the support catheter 124 over the guidewire 120 and reduces the likelihood that the guidewire 120 will back out during insertion of the support catheter 124 into the occlusion.

After the support catheter 124 has been inserted at least partially into the occlusion 104, it may be desirable to expand the support catheter's expandable member 130 into the occlusion 104 and retract the guidewire's expandable member 134 from the occlusion, thereby locking the support catheter 124 within the occlusion, according to steps 1135 and 1140 of FIG. 11 and as depicted in FIG. 6. After the support catheter's expandable member 130 is expanded into the occlusion 104, the clinician may further insert the guidewire 120 through the support catheter 124 and into the occlusion 104, according to step 1145 of FIG. 11. Expanding the support catheter's expandable member 130 into the occlusion 104 assists in holding the support catheter 124 in place during insertion of the guidewire 120 through the support catheter 124 and reduces the likelihood that the support catheter 124 will back out during insertion of the guidewire 120 into the occlusion and the support catheter 124 increases the guidewire's rigidity and column strength as it is inserted further into the occlusion 104.

After the guidewire 120 has been inserted further into and/or though the occlusion 104, it may be desirable to retract the support catheter's expandable member 130 from the occlusion 104 so that the support catheter 124 can be removed from the patient's vasculature according to step 1150 of FIG. 11. If the guidewire 120 has not crossed the occlusion 104, then it may be desirable repeat, and if necessary continue to repeat, steps 1125 through 1145 until the guidewire 120 crosses the occlusion. Once the guidewire 120 has crossed the occlusion 104, the support catheter's expandable member 130 is retracted (assuming it is expanded into the occlusion 104) from the patient's vasculature 108. After the support catheter 124 has been removed from the patient's vasculature 108, an atherectomy device may be inserted into the patient's vasculature 108 over the guidewire 120 and the occlusion, or a portion thereof, may be removed using the atherectomy device, according to step 1155 of FIG. 11

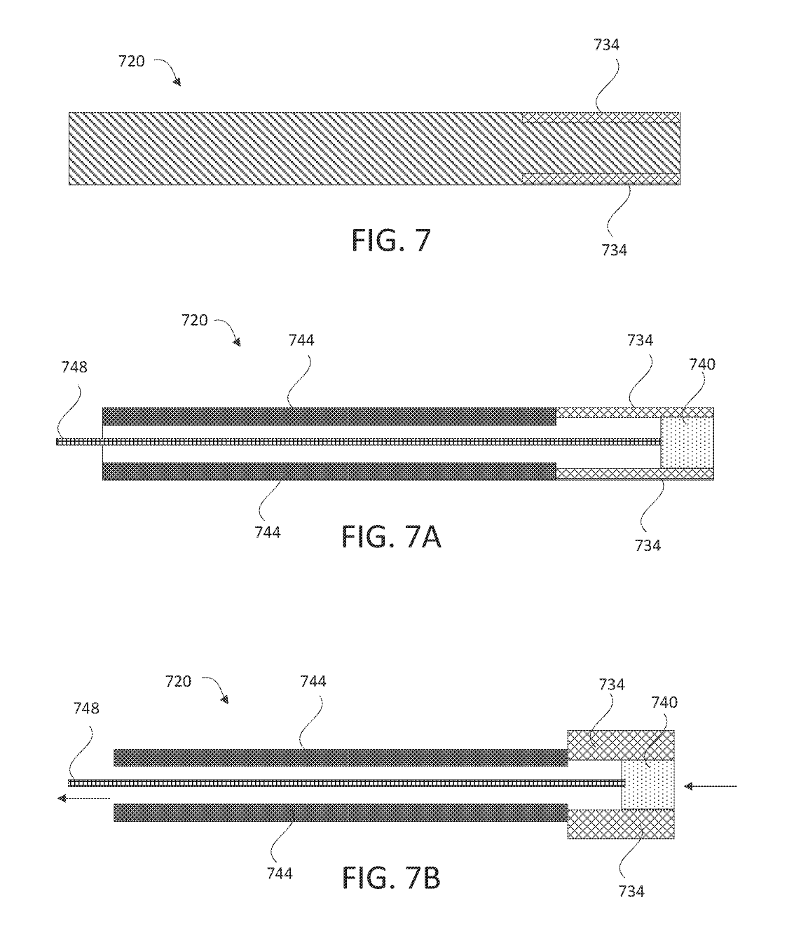

Referring to FIG. 7, there is depicted a guidewire 720, according to one embodiment of the present disclosure, wherein the guidewire 720 includes an expandable member 734 in an unexpanded state. Referring to FIG. 7A, there is depicted a cross-sectional view of the guidewire 720 of FIG. 7. As illustrated in FIG. 7A, the guidewire 720 includes an outer mandrel (or tubular sheath) 744 and an inner mandrel (or rod) 748 that is slidable with respect to the outer mandrel 744. The guidewire 720 may also include a distal tip or element 740. Opposite ends of the expandable member 734 are coupled to the distal end of the outer mandrel 744 and the distal tip 740. Accordingly, when the inner mandrel 748 is translated distally with respect to the outer mandrel 744, the expandable member 734 moves into a retracted or un-expanded state, as illustrated in FIG. 7A. And when the inner mandrel 748 translates proximally with respect to the outer mandrel 744, the expandable member 734 moves into an expanded state, as illustrated in FIG. 7B.

The expandable member 734 may be constructed of various biocompatible metals (e.g., stainless steel) and polymers. The expandable member 734 may include expandable braided element, an expandable coiled wire, or a radially expandable hypotube, and expandable ribbon. Although this embodiment of the expandable member and its movement is described and illustrated with respect to the guidewire 120, this embodiment and its movement may also be utilized with the support catheter 124.

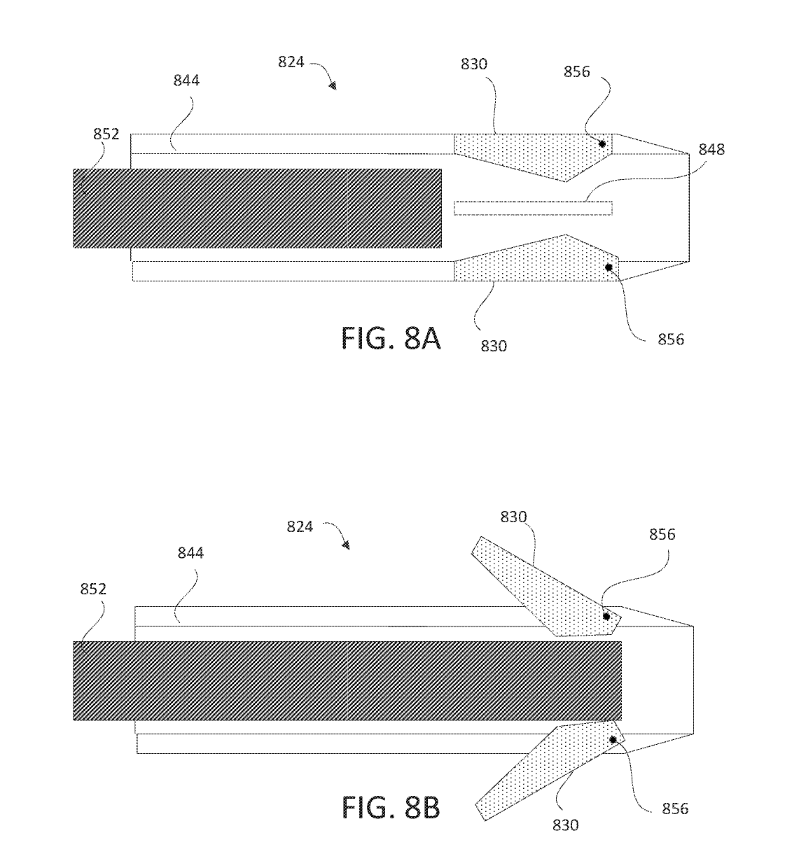

Referring to FIGS. 8A and 8B, there is depicted a support catheter 824, according to one embodiment of the present disclosure, wherein the support catheter 824 includes an outer mandrel (or tubular sheath) 844 and an inner mandrel (or rod) 852 disposed radially within the support catheter 824 and slidable. The support catheter 824 may also include expandable members 830, which are coupled to the outer mandrel 844 via pins 856, which allow the expandable members 830 to rotate thereabout. The support catheter 824 has a plurality of slots 848 that correspond to the plurality of expandable members 830, which pass through the slots upon extension. As the expandable members 830 rotate radially, they move from an unexpanded or unextended state, as depicted in FIG. 8A, to an expanded or extended state, as depicted in FIG. 8B, and extend through the slots 848. The expandable members 830 depicted in FIGS. 8A and 8B include spring-action pivotable barbs. Accordingly, when the inner mandrel 852 located proximally of the expandable members 834, the expandable members 834 remain within the outer mandrel 844. When the inner mandrel 852 translates distally and contacts the expandable members 834, the expandable members 834 rotate about the pins 856 move into an extended or un-expanded state, as illustrated in FIG. 8B. Because the pins 856 and/or the expandable members 834 are spring actuated, when the inner mandrel 852 translates proximally with respect to the outer mandrel 844, the spring-actuation causes the expandable members 834 to move back into an extended state, as illustrated in FIG. 8A. Although this embodiment of the expandable member and its movement is described and illustrated with respect to the support catheter, this embodiment and its movement may also be utilized with the guidewire.

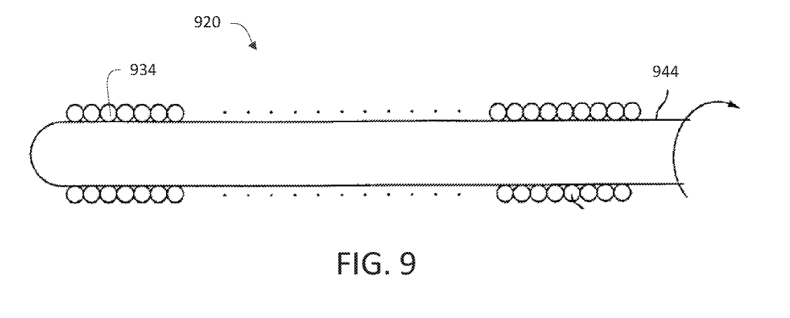

Referring to FIG. 9, there is depicted another embodiment of an expandable member of the present disclosure that can be incorporated in either a support catheter or a guidewire. The guidewire 920, according to this embodiment, has a mandrel 944 with a coil 934 wrapped therearound. The coil 934 is preferably a metal wire, such as a stainless steel wire. The guidewire 920 has a tightly wrapped configuration with an outer diameter less than the inner lumen diameter of the support catheter and/or a patient's vasculature. After the surgeon inserts the guidewire 920 into the occlusion, the surgeon rotates the mandrel 944 about a longitudinal axis so as to cause the coil 934 to partially unwind, thus obtaining a loosely wound configuration with an increased diameter. The coil 934 in the loosely wound configuration locks onto the occlusion. The surgeon can then apply force to the support catheter and insert the support catheter into the occlusion. The expandable member 934 can be retracted by rotating the mandrel in a direction to cause the coil 934 to wind more tightly, thus obtaining a tightly wound configuration. The surgeon then can further advance into and/or remove the guidewire 920 from the support catheter and/or the occlusion. Again, although this embodiment of the expandable member and its movement is described and illustrated with respect to the guidewire 920, this embodiment and its movement may also be utilized with the support catheter.

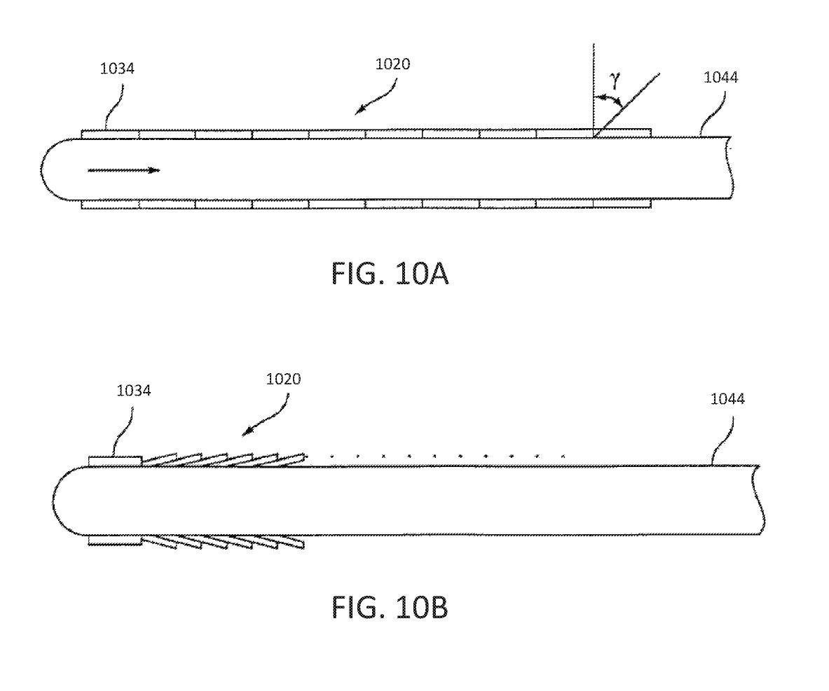

FIGS. 10A and 10B show a further embodiment of a guidewire 1020 according to the present disclosure. The guidewire 920, according to this embodiment, has a mandrel 1044 and a helical ribbon 1034 wrapped around the mandrel 1044. The most distal portion of the helical ribbon 1034 is attached to the mandrel 1044, preferably by adhesive or welding. The interface between adjacent portions of the helical ribbon 1034 preferably has an up-slope from the distal end to the proximal end of the mandrel which is an acute angle γ. Preferably, the angle between all adjacent portions of the ribbon meets at a substantially uniform angle γ. In operation, the surgeon inserts the guidewire 1020 into inner lumen diameter of the support catheter and/or a patient's vasculature. The surgeon applies traction to the mandrel 1044 which causes the helical ribbon 1034 to partially overlap itself, as illustrated in FIG. 10B. The outer diameter of the guidewire 1020 in the configuration illustrated in FIG. 10B is larger than that illustrated in FIG. 10A. Consequently, the guidewire 1020 expands into and locks into the occlusion. The expandable member 1034 can be retracted by releasing the traction applied to the mandrel or reversion the traction force applied to the mandrel in a direction to cause the helical ribbon 1034 to return to the configuration illustrated in FIG. 10A. The surgeon then can further advance into and/or remove the guidewire 1020 from the support catheter and/or the occlusion. Again, although this embodiment of the expandable member and its movement is described and illustrated with respect to the guidewire 1020, this embodiment and its movement may also be utilized with the support catheter.

The present disclosure, in various aspects, embodiments, and configurations, includes components, methods, processes, systems and/or apparatus substantially as depicted and described herein, including various aspects, embodiments, configurations, sub combinations, and subsets thereof. For example, although the present disclosure discusses a kit and a method of using a kit, wherein the kit comprises a support catheter and a guidewire, the support catheter and guidewire may be used separately. Additionally, the support catheter and guidewire may be used individually in conjunction with other devices. For instance, the guidewire of the present disclosure may be used with a laser catheter without the support catheter. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A kit comprising:
a catheter having a proximal end and a distal end, wherein the catheter comprises an expandable member adjacent its distal end; and
a guidewire having a proximal end and a distal end, wherein the guidewire is configured to be disposed radially within the catheter, wherein the guidewire comprises an expandable member adjacent its distal end.

2. The kit of claim 1, wherein the expandable member of the catheter comprises at least one of an expandable braided element, an expandable coiled wire, pivotable barbs, a radially expandable hypotube, and expandable ribbon.

3. The kit of claim 1, wherein the expandable member of the guidewire comprises at least one of an expandable braided element, an expandable coiled wire, pivotable barbs, a radially expandable hypotube, and expandable ribbon.

4. A method of crossing an occlusion within a vasculature of a subject, the method comprising:
introducing a kit within the vasculature of the subject, the kit comprising:
a catheter having a proximal end and a distal end, wherein the catheter comprises an expandable member adjacent its distal end; and
a guidewire having a proximal end and a distal end, wherein the guidewire is configured to be disposed radially within the catheter, wherein the guidewire comprises an expandable member adjacent its distal end;
positioning the guidewire and the catheter adjacent the occlusion within the vasculature;
advancing the guidewire into the occlusion;
expanding the expandable member of the guidewire into the occlusion;
advancing the catheter into the occlusion over the guidewire;
expanding the expandable member of the catheter into the occlusion;
retracting the expandable member of the guidewire from the occlusion; and
advancing the guidewire through the occlusion.

5. The method of claim 4, further comprising the step of removing the catheter from the vasculature.

6. The method of claim 4, wherein the expandable member of the catheter comprises at least one of an expandable braided element, an expandable coiled wire, pivotable barbs, a radially expandable hypotube, and expandable ribbon.

7. The method of claim 4, wherein the expandable member of the guidewire comprises at least one of an expandable braided element, an expandable coiled wire, pivotable barbs, a radially expandable hypotube, and expandable ribbon.

8. A method of removing a portion of an occlusion within a vasculature of a subject, the method comprising:
introducing a kit within the vasculature of the subject, the kit comprising:
a catheter having a proximal end and a distal end, wherein the catheter comprises an expandable member adjacent its distal end; and
a guidewire having a proximal end and a distal end, wherein the guidewire is configured to be disposed radially within the catheter, wherein the guidewire comprises an expandable member adjacent its distal end;
positioning the guidewire and the catheter adjacent the occlusion within the vasculature;
advancing the guidewire into the occlusion;
expanding the expandable member of the guidewire into the occlusion;
advancing the catheter into the occlusion over the guidewire;
expanding the expandable member of the catheter into the occlusion;
retracting the expandable member of the guidewire from the occlusion;
advancing the guidewire through the occlusion;
removing the catheter from the vasculature;
introducing an atherectomy device into the vasculature over the guidewire and removing the portion of the occlusion from the vasculature with the atherectomy device.

9. The method of claim 8, wherein the atherectomy device is a laser ablation catheter.

10. The method of claim 8, wherein the expandable member of the catheter comprises at least one of an expandable braided element, an expandable coiled wire, pivotable barbs, a radially expandable hypotube, and expandable ribbon.

11. The method of claim 8, wherein the expandable member of the guidewire comprises at least one of an expandable braided element, an expandable coiled wire, pivotable barbs, a radially expandable hypotube, and expandable ribbon.
